(19) United States
(12) Patent Application Publication
(10) Pub. No.: US 2009/0082848 A1
(43) Pub. Date: Mar. 26, 2009

(54) STENT

(76) Inventors: Koji Mori, Okayama (JP); Shuzou Yamashita, Okayama (JP)

Correspondence Address: GREENBLUM & BERNSTEIN, P.L.C., 1950 ROLAND CLARKE PLACE, RESTON, VA, 20191, US

(21) Appl. No.: 12/090,909

(22) PCT Filed: Oct. 26, 2006
(86) PCT No.: PCT/JP2006/321922

§ 371 Date: Apr. 21, 2008

(30) Foreign Application Priority Data
Oct. 31, 2005 (JP) 2005-316060

(51) Int. Cl. A61F 2/86

(52) U.S. Cl. 623/1.15

(57) ABSTRACT

A stent includes a plurality of first portions arranged in a longitudinal direction, and second portions each disposed between the adjacent first portions. Each of the first portions is expandable and contractible in a radial direction, and each of the second portions allows a whole of the stent to curve in the longitudinal direction. When the first portion is expanded in the radial direction, the longitudinal length of the first portion is decreased, but spacing between the adjacent first portions is increased.

### TECHNICAL FIELD

This invention relates to a stent for use as an instrument for treatment of stenotic symptoms of human tubular organs, such as the coronary arteries, the biliary tract, and the arteries of the head and neck.

### BACKGROUND ART

As is well known among people skilled in the art, an instrument, called a stent, is used as a therapeutic instrument for stenotic symptoms of human tubular organs. Such a stent needs to be transported to a required site through a human's curved tubular organ. Thus, it is important for the stent to be easily bendable in a longitudinal direction. It is also important that when the stent after transportation to the required site is expanded in a radial direction to correct stenosis or the like of the tubular organ, the stent can fully withstand a radially inward force, exerted from the tubular organ, namely, a force about to contract the stent in the radial direction, and can maintain the tubular organ in a required state.

Japanese Patent No. 3654627 discloses a typical example of a stent which can fulfill the above two requirements. This stent is of a cylindrical shape as a whole, is formed integrally overall, and includes a plurality of first portions arranged in the longitudinal direction, and second portions each disposed between the adjacent first portions. The first portion is composed of struts extending in a circumferential direction in a zigzag form, and is expandable and contractible in the radial direction. The second portion is composed of a plurality of struts which are arranged with spacing in the circumferential direction, and which are each S-shaped in the longitudinal direction, each of the struts being connected at both ends to the struts of the first portions located on both sides of the strut of the second portion. The second portion allows the stent to bend or curve in the longitudinal direction sufficiently easily.

The stent disclosed in the above-mentioned Japanese Patent No. 3654627 is an excellent one which can fulfill the aforementioned two requirements. However, this stent is still not fully satisfactory, and has the following problems to be solved: First, when the stent transported to the site of the tubular organ to be treated is expanded in the radial direction, the longitudinal length of the stent is reduced. This reduction phenomenon of the stent is generally called “a shortening phenomenon”. With further reference to this point, when treating the site of stenotic symptoms in the tubular organ, for example, a doctor transports the stent while observing a picture of the tubular organ, and expands the stent in the radial direction at the required site. At this time, it is important that the stent be positioned with sufficient precision at the required site. In positioning the stent, it is not necessarily easy to select the position of the stent in expectation of the longitudinal shortening of the stent along with the radial expansion of the stent. Thus, it is highly likely that an operating error will occur. To improve operability by the doctor and avoid the occurrence of the misoperation, it is desired to minimize the longitudinal shortening of the stent associated with the radial expansion of the stent. Secondly, in the stent disclosed in the above Japanese Patent No. 3654627, the first portion has the function of supporting the tubular organ and maintaining it in the required state. On the other hand, the second portion located between the first portions does not have the function of supporting the tubular organ and maintaining it in the required state. Thus, it is impossible to maintain, without fail, the required state sufficiently uniformly throughout the required site of the tubular organ.

### DISCLOSURE OF THE INVENTION

It is a principal object of the present invention to provide a novel and improved stent which can avoid the shortening or contraction of the stent in the longitudinal direction when the stent is expanded in the radial direction.

It is another object of the present invention to provide a novel and improved stent which can effect the function of supporting the tubular organ by the second portion as well as the first portion to maintain the tubular organ in the required state, in addition to the attainment of the above principal object.

Other technical objects of the present invention will become clear from descriptions to be offered later for illustrating in detail the preferred embodiments of the stent constituted in accordance with the present invention.

The inventors have found, upon diligent studies, that the above-mentioned principal object can be attained by constituting the stent in the following manner: When the first portion expandable and contractible in the radial direction is expanded in the radial direction, the longitudinal length of the first portion decreases, while spacing between this first portion and the adjacent first portion increases.

The inventors have further found that the other technical objects can be attained by annexing coupling struts, which are expandable and contractible in the radial direction, to the second portion.

According to the present invention, as a stent for attaining the above principal object, there is provided a stent cylindrical in shape as a whole and formed integrally overall, including a plurality of first portions arranged in a longitudinal direction, and second portions each disposed between the adjacent first portions, each of the first portions being expandable and contractible in a radial direction, and each of the second portions allowing a whole of the stent to curve in the longitudinal direction, wherein when the first portion is expanded in the radial direction, a longitudinal length of the first portion is decreased, but spacing between the adjacent first portions is increased.

Preferably, a tilting strut is included which, when the first portion is expanded in the radial direction, tilts in accordance with the radial expansion, and tilting of the tilting strut results in an increase in the spacing between the adjacent first portions. It is preferred that positions in a circumferential direction of two of the tilting struts adjacent in the longitudinal direction of the stent be displaced from each other. In a preferred embodiment, a first additional strut is connected to one end of the tilting strut; a second additional strut is connected to other end of the tilting strut; the tilting strut, the first additional strut, and the second additional strut constitute an S-element forming an S-shape in the circumferential direction; and when the first portion is expanded in the radial direction, the tilting strut tilts in a direction where an inclination angle of the tilting strut with respect to a longitudinal axis of the stent increases, whereby the spacing between the adjacent first portions increases. Preferably, a plurality of the S-elements are disposed in the second portion at spaced intervals in the circumferential direction; a coupling strut is disposed for coupling the first additional strut of one of the S-elements adjacent in the circumferential direction to the second additional strut of other of the adjacent S-elements, and rigidity of the coupling strut is greater than rigidity of the tilting strut, the first additional strut, and the second additional strut. It is preferred that one end of the coupling strut be connected to an intermediate part of the first additional strut, and other end of the coupling strut be connected to an intermediate part of the second additional strut. The other object of the invention described above is attained by rendering the coupling strut expandable and contractible in the radial direction. Preferably, the first portion includes a plurality of first inclined struts and a plurality of second inclined struts; the first inclined struts and the second inclined struts are arranged alternately in the circumferential direction; the first inclined strut extends from one end to other end in such a manner as to be inclined to one side in the circumferential direction at an inclination angle α with respect to the longitudinal axis of the stent; the second inclined strut extends from one end, which is connected to the other end of the first inclined strut, in a direction opposite to the first inclined strut in such a manner as to be inclined to one side in the circumferential direction at an inclination angle β with respect to the longitudinal axis of the stent, and other end of the second inclined strut is connected to the one end of the next first inclined strut; rigidity of the first inclined strut and the second inclined strut constituting the first portion is greater than rigidity of the tilting strut, the first additional strut, and the second additional strut constituting the S-element; and the S-element is located between closest sites of the first portions adjacent in the longitudinal direction, and is connected between a junction of the other end of the first inclined strut and the one end of the second inclined strut and a junction of the one end of the first inclined strut and the other end of the second inclined strut, the latter junction being located in proximity to the former junction.

In still another preferred embodiment, a plurality of the tilting struts are disposed in the first portion at spaced intervals in the circumferential direction, and the first additional strut and the second additional strut constitute the second portions. Preferably, the first portion includes a plurality of first inclined struts and a plurality of second inclined struts; the first inclined struts and the second inclined struts are arranged alternately in the circumferential direction, except the tilting strut connected between the first inclined struts or between the second inclined struts; the first inclined strut extends from one end to other end in such a manner as to be inclined to one side in the circumferential direction at an inclination angle α with respect to the longitudinal axis of the stent; the second inclined strut extends from one end, which is connected to the other end of the first inclined strut, in a direction opposite to the first inclined strut in such a manner as to be inclined to one side in the circumferential direction at an inclination angle β with respect to the longitudinal axis of the stent, and other end of the second inclined strut is connected to the one end of the next first inclined strut; and rigidity of the first inclined strut and the second inclined strut constituting the first portion is greater than rigidity of the tilting strut, the first additional strut, and the second additional strut constituting the S-element.

In a further preferred embodiment, the tilting strut has a middle part disposed in the first portion, and opposite end portions connected to opposite ends of the middle part and constituting the second portions; and when the first portion is expanded in the radial direction, the tilting strut tilts in a direction where an inclination angle of the tilting strut with respect to a longitudinal axis of the stent decreases, whereby the spacing between the adjacent first portions increases. It is preferred that rigidity of at least the opposite end portions of the tilting strut be lower than rigidity of other struts constituting the first portion.

According to the stent of the present invention, when the first portion expandable and contractible in the radial direction is expanded in the radial direction, the longitudinal length of the first portion decreases, but spacing between the adjacent first portions increases. Thus, it is possible to avoid the longitudinal contraction of the stent during the radial expansion of the stent.

In the shape of the stent of the present invention in which the coupling strut expandable and contractible in the radial direction is annexed to the second portion, it is possible to show the function of supporting the tubular organ by the second portion as well as the first portion, thereby maintaining the required unclosed state.

Stents are roughly classified into two types, a stent of a type which is transported to a required site of a tubular organ while being contracted in the radial direction, as desired, and in which a balloon catheter located within the stent is inflated at the required site to deform the stent plastically until expansion in the radial direction (plastic expansion type stent); and a stent of a type which is elastically contracted in the radial direction, accommodated into a sheath, and transported to a required site of a tubular organ, where the sheath is separated to restore the stent elastically into the original state and expand it in the radial direction (elastic expansion type stent). The present invention can be applied to any of the types.

### BRIEF DESCRIPTION OF THE DRAWINGS

FIG. 1 is a front view showing a preferred embodiment of a stent constituted in accordance with the present invention.

FIG. 2 is a developed view showing a part of the stent of FIG. 1 developed in the circumferential direction.

FIG. 3 is an enlarged developed partial view showing a part of the stent of FIG. 1 on an enlarged scale.

FIG. 4 is an enlarged developed partial view, similar to FIG. 3, showing the stent of FIG. 1 contracted in the radial direction.

FIG. 5 is an enlarged developed partial view, similar to FIG. 3, showing the stent of FIG. 1 expanded in the radial direction.

FIG. 6 is a developed view showing a part of another embodiment of a stent, constituted in accordance with the present invention, as developed in the radial direction.

FIG. 7 is an enlarged developed partial view showing a part of the stent of FIG. 6 on an enlarged scale.

FIG. 8 is an enlarged developed partial view, similar to FIG. 7, showing the stent of FIG. 6 contracted in the radial direction.

FIG. 9 is an enlarged developed partial view, similar to FIG. 8, showing the stent of FIG. 6 expanded in the radial direction.

FIG. 10 is an enlarged developed partial view showing a part of still another embodiment of a stent, constituted in accordance with the present invention, on an enlarged scale.

FIG. 11 is an enlarged developed partial view, similar to FIG. 10, showing the stent of FIG. 10 contracted in the radial direction.

FIG. 12 is an enlarged developed partial view, similar to FIG. 10, showing the stent of FIG. 10 expanded in the radial direction.

FIG. 13 is an enlarged developed partial view, similar to FIG. 10, showing a modification of the stent of FIG. 10.

### BEST MODE FOR CARRYING OUT THE INVENTION

Preferred embodiments of the stent constituted in accordance with the present invention will be described in further detail by reference to the accompanying drawings.

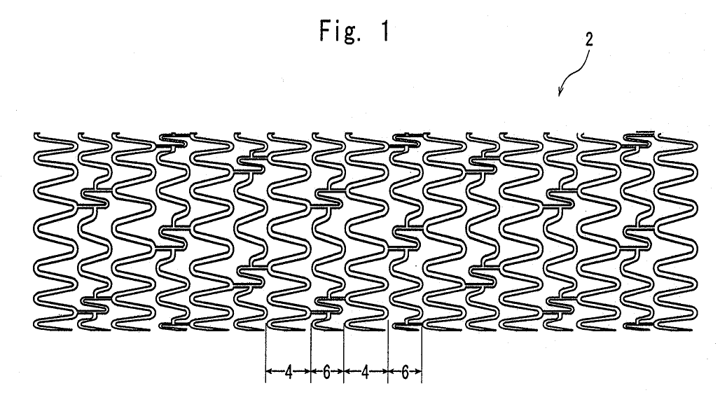

FIG. 1 shows a preferred embodiment of a plastic expansion type stent constituted in accordance with the present invention. A stent, entirely indicated at the numeral 2, is of a cylindrical shape as a whole, and is formed integrally overall. Such a stent 2 can be produced advantageously by applying a laser beam to a cylindrical body to remove sites other than a required site and, where necessary, further performing polishing such as electrolytic polishing or chemical polishing. The cylindrical body is preferably one formed from a suitable metal such as stainless steel or a cobalt-chromium (CoCr) alloy. A suitable synthetic resin coating having biocompatibility can be applied to the surface of the stent 2. If desired, a required chemical can also be applied to it.

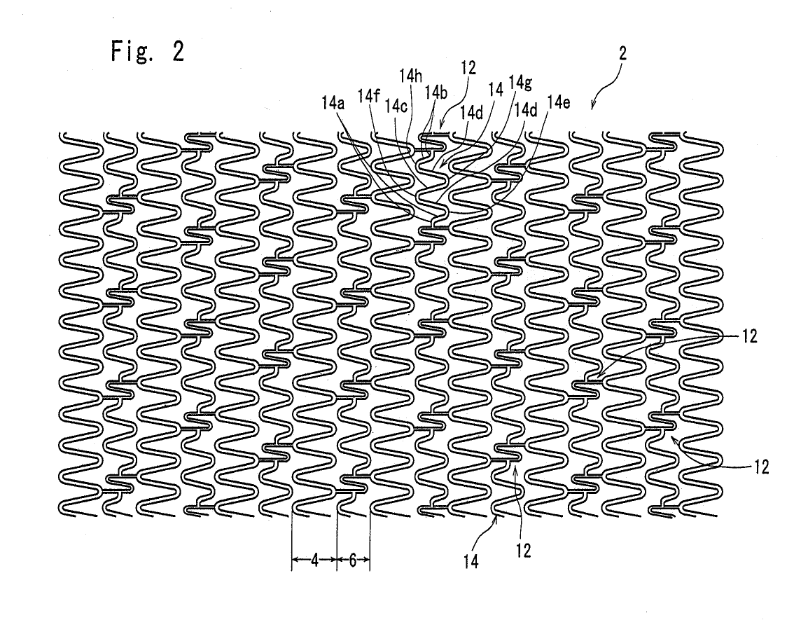

As will be clearly understood from FIG. 1 and FIG. 2 showing a part of the stent, shown in FIG. 1, as developed in the circumferential direction, the illustrated stent 2 includes a plurality of first portions 4 arranged in the longitudinal direction, namely, in the right-and-left direction in FIGS. 1 and 2, and a plurality of second portions 6 each of which is disposed between the adjacent first portions 4, 4.

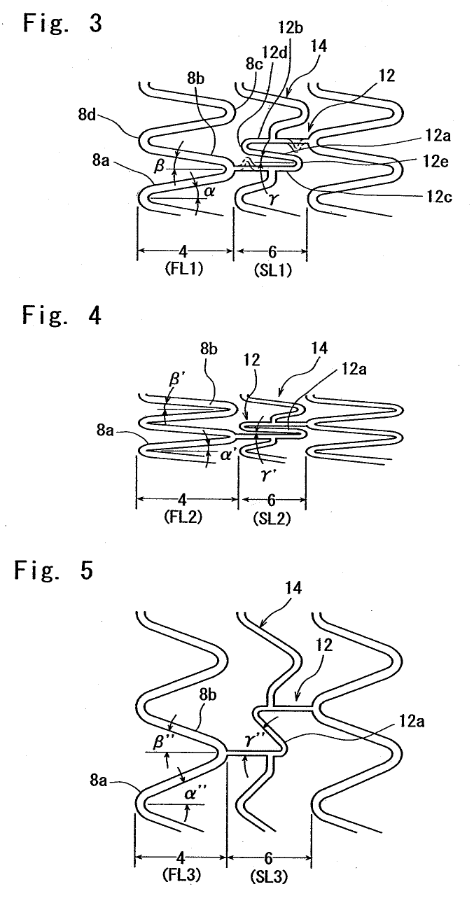

With further reference to FIG. 3 showing a part of FIG. 2 on an enlarged scale, the first portion 4 is composed of struts extending in a zigzag form in the circumferential direction, namely, in the up-and-down direction in FIGS. 2 and 3. In further detail, the first portion 4 includes a plurality of first inclined struts 8a and a plurality of second inclined struts 8b, and the first inclined struts 8a and the second inclined struts 8b are arranged alternately in the circumferential direction. The first strut 8a extends from the left end (i.e., one end) to the right end (i.e., the other end) in FIG. 3 substantially straightly in an upwardly inclined manner (i.e., in a manner inclined to one side in the circumferential direction) at an inclination angle α with respect to the longitudinal axis of the stent 2. The second strut 8b extends from the right end (i.e., one end) to the left end (i.e., the other end) in FIG. 3 substantially straightly in an upwardly inclined manner (i.e., in a manner inclined to one side in the circumferential direction) at an inclination angle α with respect to the longitudinal axis of the stent 2. It is generally advantageous for the inclination angle α and the inclination angle β to be substantially the same. However, these inclination angles may be different from each other and, preferably, are of the order of 5 to 20 degrees. The other end or the right end of the first inclined strut 8a, and the one end or the right end of the second inclined strut 8b are connected via a semi-arcuate connecting portion 8c. The other end or the left end of the second inclined strut 8b is connected to one end or the left end of a next first inclined strut 8a (i.e., the one located above the second inclined strut 8b) via a semi-arcuate connecting portion 8d. If desired, instead of allowing the first strut 8a and/or the second strut 8b to extend straightly, the first strut 8a and/or the second strut 8b can be formed into a suitable shape having a suitable bending portion, as disclosed in the aforementioned Japanese Patent No. 3654627. Moreover, the first inclined strut 8a and the second inclined strut 8b can be connected via connecting portions of other suitable shape in place of the semi-arcuate connecting portions 8c and 8d.

With further reference to FIGS. 1 to 3, the second portion 6 has a plurality of S-shaped S-elements 12 disposed with spacing in the circumferential direction. The S-element 12 is composed of a tilting strut 12a and additional struts 12b and 12c. The left end or one end of the additional strut 12b is connected to the left end or one end of the tilting strut 12a via a semi-arcuate connecting portion 12d, and the additional strut 12b extends from the one end toward the other end of the tilting strut 12a, namely, rightward in FIG. 3. On the other hand, one end or the right end of the additional strut 12c is connected to the other end or right end of the tilting strut 12a via a semi-arcuate connecting portion 12e, and the additional strut 12c extends from the one end toward the one end of the tilting strut 12a, namely, leftward in FIG. 3. The tilting strut 12a preferably extends substantially parallel to, or at an inclination angle γ of the order of more than 0, but less than 30 degrees (i.e., 0<γ<30 degrees) with respect to, the longitudinal direction of the stent 2. The tilting struts 12b and 12c also preferably each extend substantially parallel to, or at an inclination angle γ of the order of more than 0, but less than 30 degrees (i.e., 0<γ<30 degrees) with respect to, the longitudinal direction of the stent 2. Such an S-element 12 is located between the closest sites of the first portions 4 located on both sides of the S-element 12, namely, between the connecting portion 8c in the first portion 4 located on the left side of the S-element 12 and the connecting portion 8d in the first portion 4 located on the right side of the S-element 12 in FIG. 3, with the right end or the other end of the additional strut 12b being connected to the connecting portion 8d, and the left end or the other end of the additional strut 12c being connected to the connecting portion 8c. The illustrated stent 2 is formed of a cylindrical body having substantially the same thickness, whereas the tilting strut 12a, the additional struts 12b and 12c, and the connecting portions 12d and 12e constituting the S-element 12 have a width which is set to be smaller than the width of the first inclined strut 8a, the second inclined strut 8b, and the connecting portions 8c and 8d in the first portion 4. Thus, the cross-sectional area of each of the tilting strut 12a, the additional struts 12b and 12c, and the connecting portions 12d and 12e constituting the S-element 12 is set to be smaller than the cross-sectional area of each of the first inclined strut 8a, the second inclined strut 8b, and the connecting portions 8c and 8d in the first portion 4, and the rigidity of the tilting strut 12a, the additional struts 12b and 12c, and the connecting portions 12d and 12e constituting the S-element 12 is lower than the rigidity of the first inclined strut 8a, the second inclined strut 8b, and the connecting portions 8c and 8d in the first portion 4. In order to render the rigidity of the tilting strut 12a, the additional struts 12b and 12c, and the connecting portions 12d and 12e constituting the S-element 12 lower than the rigidity of the first inclined strut 8a, the second inclined strut 8b, and the connecting portions 8c and 8d in the first portion 4, it is possible, if desired, to decrease the thickness instead of, or in addition to, rendering the width small.

In the S-element 12 in the illustrated embodiment, the left end of the additional strut 12b located above the tilting strut 12a is connected to the left end of the tilting strut 12a, and the right end of the additional strut 12c located below the tilting strut 12a is connected to the right end of the tilting strut 12a. Instead, however, the right end of the additional strut located above the tilting strut can be connected to the right end of the tilting strut, and the left end of the additional strut located below the tilting strut can be connected to the left end of the tilting strut; accordingly, the element composed of the tilting strut and the two additional struts can form a mirror image of an S (i.e., a Z) obtained when the letter S is reflected in a mirror. Thus, the term “S” or “letter S”, as used herein, includes not only an ordinary letter S, but also a mirror image of S (i.e., letter Z). In the illustrated embodiment, moreover, the additional struts 12b and 12c extend substantially straightly. If desired, however, a part of each of the additional struts 12b and 12c can be formed in a U-shape or an inverted U-shape in the circumferential direction, or in an S-shape in the longitudinal direction, as shown, for example, by dashed double-dotted lines in FIG. 3, in order for the longitudinal expansion and contraction of the second portion 6 to be achieved more easily, accordingly, in order for the stent 2 to be curved in the longitudinal direction more easily. Furthermore, the tilting strut 12a and the additional struts 12b and 12c can be connected by connecting portions of other suitable shape instead of the semi-arcuate connecting portions 12d and 12e.

In the illustrated embodiment, coupling struts 14 for coupling the S-elements 12 disposed with spacing in the circumferential direction are disposed in the second portion 6. The illustrated coupling strut 14 extends in the circumferential direction in a zigzag form. As will be clearly understood from FIG. 2, the illustrated coupling strut 14 has a lower end portion or one end portion 14a extending upwardly in FIG. 2 from an intermediate portion of the additional strut 12b of the S-element 12, and then extending rightward in an upwardly inclined manner, and an upper end portion or the other end portion 14b extending downwardly in FIG. 2 from an intermediate portion of the additional strut 12c of the next (i.e., upper) S-element 12, and then extending leftward in a downwardly inclined manner. The lower end of the one end portion 14a and the upper end of the other end portion 14b of the coupling strut 14 are preferably connected to the additional struts 12b and 12c, respectively, of the S-elements nearly perpendicularly, but can be connected to the additional struts 12b and 12c, respectively, of the S-elements at a suitable inclination angle. One first inclined strut 14c extending rightward in an upwardly inclined manner, and second inclined struts 14d arranged above and below the first inclined strut 14c and extending leftward in an upwardly inclined manner are disposed between the one end portion 14a and the other end portion 14b. The upper end of the one end portion 14a and the right end of the second inclined strut 14d are connected by a semi-arcuate connecting portion 14e, the left end of the second inclined strut 14d and the left end of the first inclined strut 14c are connected by a semi-arcuate connecting portion 14f, the right end of the first inclined strut 14c and the right end of the second inclined strut 14d are connected by a semi-arcuate connecting portion 14g, and the left end of the second inclined strut 14d and the lower end of the other end portion 14b are connected by a semi-arcuate connecting portion 14h. Preferably, the width of the one end portion 14a, the other end portion 14b, the first inclined strut 14c, the second inclined strut 14d, and the connecting portions 14e to 14h constituting the coupling strut 14 is substantially the same as the width of the first inclined strut 8a, the second inclined strut 8b, and the connecting portions 8c and 8d in the first portion 4, and is thus larger than the width of the tilting strut 12a, the additional struts 12b and 12c, and the connecting portions 12d and 12e constituting the S-element 12. Accordingly, the rigidity of the one end portion 14a, the other end portion 14b, the first inclined strut 14c, the second inclined strut 14d, and the connecting portions 14e to 14h constituting the coupling strut 14 is greater than the rigidity of the tilting strut 12a, the additional struts 12b and 12c, and the connecting portions 12d and 12e constituting the S-element 12. The lengths and inclination angles of the first inclined strut 14c and the second inclined strut 14d in the coupling strut 14 may be substantially the same as those of the first inclined strut 8a and the second inclined strut 8b in the first portion 4.

The actions and effects of the above-described stent 2 will be described below. In transporting the stent 2 to the site of the human tubular organ to be treated, the stent 2 is plastically deformed and contracted in the radial direction, as shown in FIG. 4. In the illustrated stent 2, the S-element 12 of the second portion 6 is located between the closest sites of the first portions 4 located on both sides of the S-element 12. Thus, when the stent 2 is contracted in the radial direction, it is not that a part of the S-element 12 is interposed between the first inclined strut 8a and the second inclined strut 8b of the first portion 4 to inhibit the first inclined strut 8a and the second inclined strut 8b from intimately contacting or approaching each other; thus, the stent 2 can be contracted sufficiently. As will be understood by comparison of and reference to FIGS. 3 and 4, when the stent 2 is contracted in the radial direction, in the first portion 4, the inclination angle α of the first inclined strut 8a decreases to α′, and the inclination angle β of the second inclined strut 8b decreases to β′. Because of these changes, the longitudinal length of the first portion 4 increases from FL1 (FIG. 3) to FL2 (FIG. 4). In the second portion 6, on the other hand, the inclination angle of the tilting strut 12a decreases from γ to γ′, so that the longitudinal length of the second portion 6 slightly decreases from SL1 (FIG. 3) to SL2 (FIG. 4). When the stent 2 is transported through the tubular organ which is curved, the S-element 12, especially, its additional struts 12b and 12c, warp appropriately, whereby the stent 2 appropriately curves in the longitudinal direction.

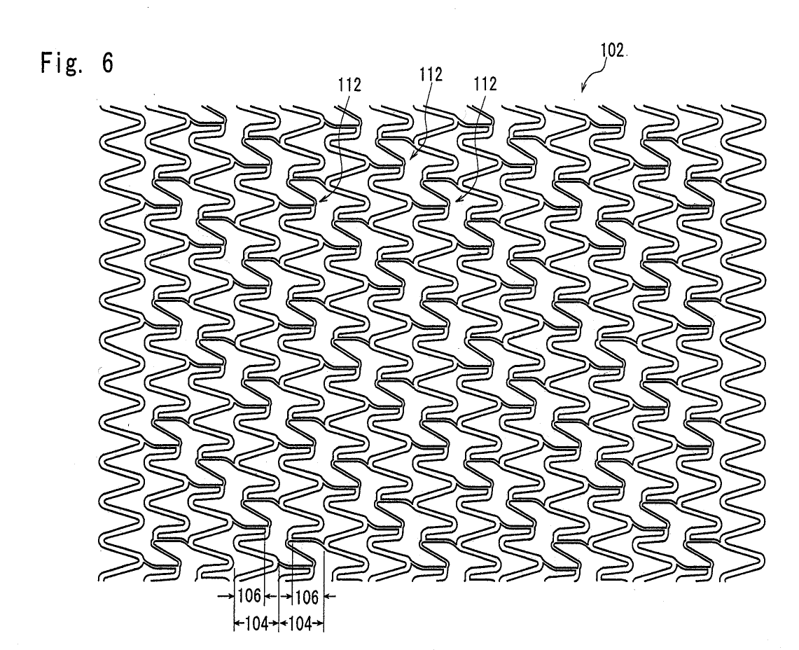

When the stent 2 has been transported to the required site of the tubular organ, the stent 2 is plastically deformed and expanded in the radial direction, as shown in FIG. 5, by inflating a balloon catheter located within the stent 2. As will be understood by reference to FIG. 5 in comparison with FIGS. 3 and 4, when the stent 2 is expanded in the radial direction, in the first portion 4, the inclination angle α of the first inclined strut 8a increases to α″, and the inclination angle β of the second inclined strut 8b increases to β″, wherefore the longitudinal length of the first portion 4 decreases to FL3 (FIG. 5). In the second portion 6, on the other hand, the inclination angle γ of the tilting strut 12a increases to γ″, so that the longitudinal length of the second portion 6 increases to SL3, whereby the spacing between the adjacent first portions 4 increases to SL3. If the decrease in the longitudinal length of the first portion 4 (i.e., FL1-FL3) is designed to be substantially equal to the increase in the longitudinal length of the second portion 6 (i.e., SL3-SL1), the longitudinal length of the stent 2 when expanded can be rendered substantially equal to the longitudinal length of the stent 2 when manufactured. If desired, the difference between the longitudinal length FL2 of the first portion 4 when the stent 2 is contracted in the radial direction and the longitudinal length FL3 of the first portion 4 when the stent 2 is expanded in the radial direction (i.e., FL2-FL3) can be designed to substantially equal the difference between the longitudinal length SL3 of the second portion 6 when the stent 2 is expanded in the radial direction and the longitudinal length SL2 of the second portion 6 when the stent 2 is contracted in the radial direction (i.e., SL3-SL2). By so doing, when the stent 2 contracted in the radial direction is transported to the required site of the tubular organ, and expanded in the radial direction at the required site, the longitudinal length of the stent 2 is maintained at its longitudinal length during transportation without being substantially changed.

In connection with the illustrated stent 2, the following facts should be further noted: When the stent 2 is transported to the required site of the tubular organ, and expanded in the radial direction, the inclination angles of the coupling strut 14 in the second portion 6, more specifically, the inclination angles of the inclined part of the one end portion 14a, the inclined part of the other end portion 14b, the first inclined strut 14c, and the second inclined strut 14d, are changed to increase the circumferential length of the coupling strut 14, as are the inclination angles of the first inclined strut 8a and the second inclined strut 8b in the first portion 4, as will be understood by reference to FIG. 5 in comparison with FIGS. 3 and 4. Hence, as does the first portion 4, the second portion 6 also maintains the tubular organ in the required state by the extended coupling strut 14, thus obtaining the function of maintaining the required state sufficiently uniformly throughout the whole of the stent 2 in the longitudinal direction.

The stent 2 explained with reference to FIGS. 1 to 5 is of a plastic expansion type, but the stent of an elastic expansion type can also have substantially the same shape applied thereto. The stent of the elastic expansion type is produced in the state illustrated in FIG. 5 which is the state expanded in the radial direction. During transport through the tubular organ, this stent is elastically contracted in the radial direction to be brought into the state illustrated in FIG. 4. In this state, the stent is accommodated in a sheath. Then, at the required site of the tubular organ, the sheath is detached from the stent to expand the stent elastically in the radial direction, thereby restoring the stent into the state illustrated in FIG. 5. The stent of the elastic expansion type can be formed advantageously, for example, from a superelastic metal such as a nickel-titanium (NiTi) alloy.

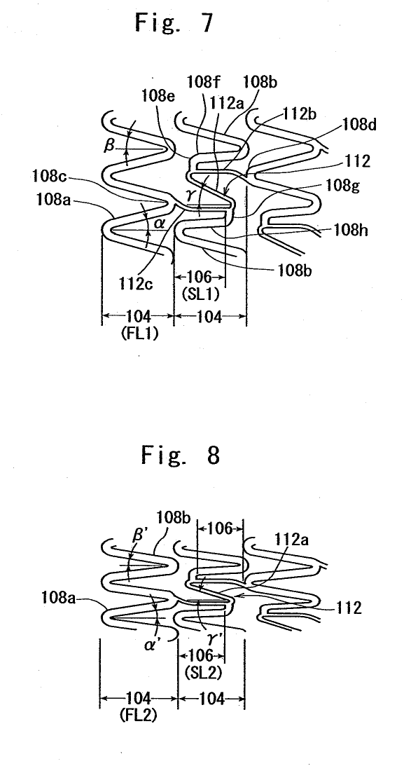

FIGS. 6 to 9 show a second embodiment of a plastic expansion type stent constituted in accordance with the present invention. With reference to FIGS. 6 and 7, a stent 102 is also of a cylindrical shape as a whole, and is formed integrally overall. A stent 102 also includes a plurality of first portions 104 arranged in the longitudinal direction, and second portions 106 each of which is disposed between the adjacent first portions 104, 104. The first portion 104 includes first inclined struts 108a and second inclined struts 108b, which are arranged alternately in the circumferential direction, as does the first portion 4 in the embodiment shown in FIGS. 1 to 5. However, with the exception of the two first portions 104 located at both ends in the longitudinal direction, the first portion 104 has a plurality of deformed portions disposed with spacing in the circumferential direction (the up-and-down direction in FIGS. 6 and 7). The deformed portion includes a tilting strut 112a. The tilting strut 112a extends from the one end or upper end thereof to the other end or lower end thereof rightward in a downwardly inclined manner at an inclination angle γ The upper end of the tilting strut 112a is connected to a second inclined strut 108b via an upwardly extending short strut 108e and an inclined strut 108f extending from the upper end of the short strut 108e rightward in a manner inclined to one side in the circumferential direction, namely, in an upwardly inclined manner. The lower end of the tilting strut 112a is connected to the second inclined strut 108b via a downwardly extending short strut 108g and an inclined strut 108h extending from the lower end of the short strut 108g leftward in a manner inclined to the other side in the circumferential direction, namely, in a downwardly inclined manner.

To the upper end and lower end of the tilting strut 112a in the first portion 104, additional struts 112b and 112c are also connected, respectively. Each of the additional struts 112b and 112c constitutes the second portion 106 located between the first portions 104, 104. The additional strut 112b extends from the upper end of the tilting strut 112a rightward substantially parallel to the longitudinal axis of the stent 102, then extends rightward in a downwardly inclined manner, and is finally connected to a connecting portion 108d at a junction between the first inclined strut 108a and the second inclined strut 108b in the adjacent first portion 104. The additional strut 112c extends from the lower end of the tilting strut 112a leftward substantially parallel to the longitudinal axis of the stent 102, then extends leftward in an upwardly inclined manner, and is finally connected to a connecting portion 108c at a junction between the first inclined strut 108a and the second inclined strut 108b in the adjacent first portion 104. The substantial whole of the additional struts 112b and 112c constituting the second portion 106 is existent in a region of extension of the first inclined strut 108a and the second inclined strut 108b constituting the first portion 104 in the longitudinal direction. In the embodiment illustrated in FIGS. 6 to 9, therefore, the second portion 106 is present overlappingly within the region of existence of the first portion 104 in the longitudinal direction.

In the embodiment shown in FIGS. 6 to 9, the tilting strut 112a and the additional struts 112b and 112c constitute an S-element 112. The width of the tilting strut 112a and the additional struts 112b and 112c constituting the S-element 112 is set to be smaller than the width of the other parts in the first portion 104. Thus, the cross-sectional area of each of the tilting strut 112a and the additional struts 112b and 112c constituting the S-element 112 is smaller than the cross-sectional area of the other parts in the first portion 104, and the rigidity of the tilting strut 112a and the additional struts 112b and 112c constituting the S-element 112 is lower than the rigidity of the other parts in the first portion 104.

In transporting the stent 102 to the site of the human tubular organ to be treated, the stent 102 is contracted in the radial direction, as shown in FIG. 8. At this time, in the first portion 104, the inclination angle α of the first inclined strut 108a decreases to α′, and the inclination angle β of the second inclined strut 108b decreases to β′. Because of these changes, the longitudinal length of the first portion 104 increases from FL1 (FIG. 7) to FL2 (FIG. 8). In the S-element 112 as well, the inclination angle of the tilting strut 112a decreases from γ to γ′.

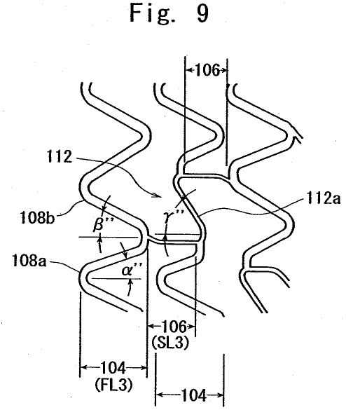

When the stent 102 has been transported to the required site of the tubular organ, the stent 102 is plastically deformed and expanded in the radial direction, as shown in FIG. 9. As will be understood by reference to FIG. 9 in comparison with FIGS. 7 and 8, when the stent 102 is expanded in the radial direction, in the first portion 104, the inclination angle α of the first inclined strut 108a increases to α″, and the inclination angle β of the second inclined strut 108b increases to β″. Moreover, the inclination angle γ of the tilting strut 112a of the S-element 112 increases to γ″. Thus, the longitudinal length of the first portion 104 decreases to FL3 (FIG. 9). As will be understood by reference to FIG. 9 in comparison with FIGS. 7 and 8, however, since the inclination angle γ of the tilting strut 112a increases to γ″, the adjacent second portions 106 are moved away from each other in the longitudinal direction, and the overlapping length of the adjacent second portions 106 in the longitudinal direction is decreased. Because of this change, the spacing between the adjacent first portions 104 is increased. Thus, the decrease in the longitudinal length of the first portion 104 is compensated for, with the result that the decrease in the longitudinal length of the entire stent 102 is avoided.

In connection with the stent 102 shown in FIGS. 6 to 9, the following facts should also be noted: As will be understood from FIG. 9, even when the stent 102 is in a radially expanded state, a considerable part of the second portion 106 in the longitudinal direction of the stent 102 exists in the region of extension of the first portion 104. In other words, the site where the first portion 104 does not exist in the longitudinal direction of the stent 102 is small. Thus, the function of maintaining the required state throughout the whole of the stent 102 in the longitudinal direction is obtained by the first portion 104.

Features, actions and effects other than the above-mentioned features, actions and effects in the embodiment shown in FIGS. 6 to 9 are substantially the same as those of the embodiment shown in FIGS. 1 to 5.

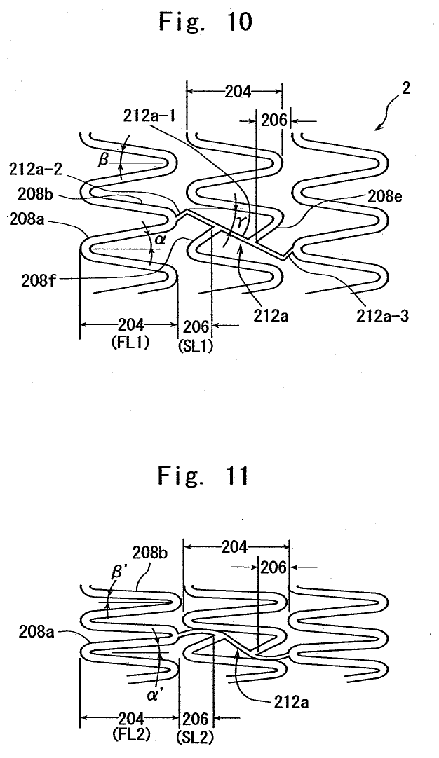

FIGS. 10 to 12 show still another embodiment of a plastic expansion type stent constituted in accordance with the present invention. A stent 202 also includes a plurality of first portions 204 arranged in the longitudinal direction, and second portions 206 each of which is disposed between the adjacent first portions 204, 204. The first portion 204 includes first inclined struts 208a and second inclined struts 208b. However, with the exception of the first portions 204 located on both sides in the longitudinal direction, the first portion 204 has a plurality of deformed portions disposed with spacing in the circumferential direction (only one of the deformed portions is shown in FIGS. 10 to 12). The deformed portion includes a tilting strut 212a. The tilting strut 212a has a main portion 212a-1 extending rightward in a downwardly inclined manner at an inclination angle γ in FIG. 10, a short one-end portion 212a-2 extending from one end or left end of the main portion leftward in a downwardly inclined manner, and a short other-end portion 212a-3 extending from the other end or right end of the main portion rightward in an upwardly inclined manner. The main portion 212a-1 is connected to the second inclined strut 208b via a strut 208e inclined rightwardly upwardly, and is also connected to the second inclined strut 208b via a strut 208f inclined leftwardly downwardly. A middle part between the site of the main portion 212a-1 of the tilting strut 212a, where the strut 208e is connected, and the site of the main portion 212a-1, where the strut 208f is connected, constitutes a part of the first portion 204. On the other hand, both side parts of the main portion 212a-1, the short one-end portion 212a-2, and the short other-end portion 212a-3 of the tilting strut 212a constitute the second portions 206.

The width of the tilting strut 212a is set to be smaller than the width of the other struts constituting the first portion 204. Thus, the cross-sectional area of the tilting strut 212a is smaller than the cross-sectional area of the other struts constituting the first portion 204, and the rigidity of the tilting strut 212a is lower than the rigidity of the other struts constituting the first portion 204. If desired, as shown in FIG. 13, the width of the middle part constituting a part of the first portion 204 in the main portion 212a-1 of the tilting strut 212a, namely, the width of the middle part between the site where the strut 208e is connected and the site where the strut 208f is connected, can be rendered larger than the width of the other parts of the tilting strut 212a to equal the width of the other parts of the first portion 204. Thus, the decrease in the rigidity in the radial direction of the first portion 204 due to the presence of the tilting strut 212a can be kept to a minimum.

In transporting the stent 202 to the site of the human tubular organ to be treated, the stent 202 is contracted in the radial direction, as shown in FIG. 11. At this time, in the first portion 204, the inclination angle α of the first inclined strut 208a decreases to α′, and the inclination angle β of the second inclined strut 208b decreases to β′. Because of these changes, the longitudinal length of the first portion 204 increases from FL1 (FIG. 10) to FL2 (FIG. 11). The tilting strut 212a deforms according to changes in the first inclined strut 208a, the second inclined strut 208b, the strut 208e, and the strut 208f.

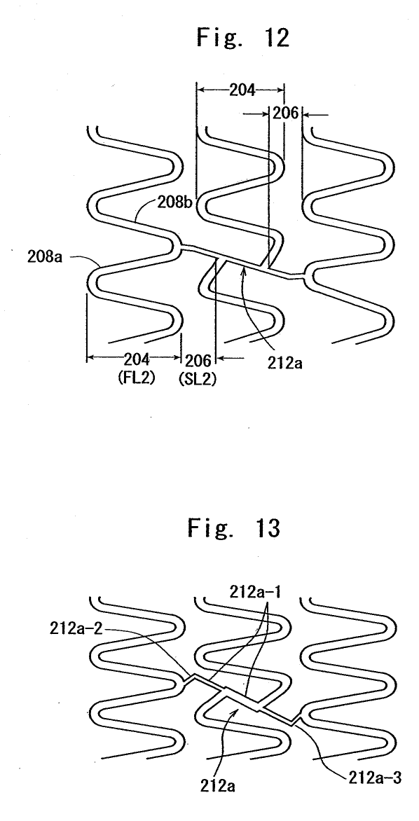

When the stent 202 has been transported to the required site of the tubular organ, the stent 202 is plastically deformed and expanded in the radial direction, as shown in FIG. 12. As will be understood by reference to FIG. 12 in comparison with FIGS. 10 and 11, when the stent 202 is expanded in the radial direction, in the first portion 204, the inclination angle α of the first inclined strut 208a increases to α″, and the inclination angle β of the second inclined strut 208b increases to β″. Thus, the longitudinal length of the first portion 204 decreases to FL3 (FIG. 12). On the other hand, the inclination angle of the main portion 212a-1 of the tilting strut 212a decreases from γ to γ″. Thus, the longitudinal length of the second portion 206 increases to SL3, and the spacing between the adjacent second portions 206 is also increased. As a result, the spacing between the adjacent first portions 204 is increased. Thus, the decrease in the longitudinal length of the first portion 204 is compensated for, so that the decrease in the longitudinal length of the entire stent 202 is avoided.

While the preferred embodiments of the stent constituted in accordance with the present invention have been described in detail by reference to the accompanying drawings, there is no need to dwell on the fact that the present invention is not limited to such embodiments, but various changes and modifications may be made without departing from the scope of the present invention. For example, instead of providing the first portion of the shape including the first inclined struts and the second inclined struts, it is permissible to constitute the first portion by arranging a plurality of circular, elliptical or polygonal parts in the circumferential direction, and to achieve the radial contraction and expansion of the first portion by the appropriate deformation of the circular, elliptical or polygonal parts.

1. A stent cylindrical in shape as a whole and formed integrally overall, including a plurality of first portions arranged in a longitudinal direction, and second portions each disposed between the adjacent first portions, each of the first portions being expandable and contractible in a radial direction, and each of the second portions allowing a whole of the stent to curve in the longitudinal direction,
wherein when the first portion is expanded in the radial direction, a longitudinal length of the first portion is decreased, but spacing between the adjacent first portions is increased.

2. The stent according to claim 1, wherein
a tilting strut is included which, when the first portion is expanded in the radial direction, tilts in accordance with the radial expansion, and
tilting of the tilting strut results in an increase in the spacing between the adjacent first portions.

3. The stent according to claim 2, wherein positions in a circumferential direction of two of the tilting struts adjacent in the longitudinal direction of the stent are displaced from each other.

4. The stent according to claim 2, wherein
a first additional strut is connected to one end of the tilting strut,
a second additional strut is connected to other end of the tilting strut,
the tilting strut, the first additional strut, and the second additional strut constitute an S-element forming an S-shape in the circumferential direction, and
when the first portion is expanded in the radial direction, the tilting strut tilts in a direction where an inclination angle of the tilting strut with respect to a longitudinal axis of the stent increases, whereby the spacing between the adjacent first portions increases.

5. The stent according to claim 4, wherein
a plurality of the S-elements are disposed in the second portion at spaced intervals in the circumferential direction,
a coupling strut is disposed for coupling the first additional strut of one of the S-elements adjacent in the circumferential direction to the second additional strut of other of the adjacent S-elements, and
rigidity of the coupling strut is greater than rigidity of the tilting strut, the first additional strut, and the second additional strut.

6. The stent according to claim 5, wherein
one end of the coupling strut is connected to an intermediate part of the first additional strut, and
other end of the coupling strut is connected to an intermediate part of the second additional strut.

7. The stent according to claim 5, wherein
the coupling strut is expandable and contractible in the radial direction.

8. The stent according to claim 5, wherein
the first portion includes a plurality of first inclined struts and a plurality of second inclined struts, the first inclined struts and the second inclined struts are arranged alternately in the circumferential direction, the first inclined strut extends from one end to other end in such a manner as to be inclined to one side in the circumferential direction at an inclination angle α with respect to the longitudinal axis of the stent, the second inclined strut extends from one end, which is connected to the other end of the first inclined strut, in a direction opposite to the first inclined strut in such a manner as to be inclined to one side in the circumferential direction at an inclination angle β with respect to the longitudinal axis of the stent, and other end of the second inclined strut is connected to the one end of the next first inclined strut,
rigidity of the first inclined strut and the second inclined strut constituting the first portion is greater than rigidity of the tilting strut, the first additional strut, and the second additional strut constituting the S-element, and
the S-element is located between closest sites of the first portions adjacent in the longitudinal direction, and is connected between a junction of the other end of the first inclined strut and the one end of the second inclined strut and a junction of the one end of the first inclined strut and the other end of the second inclined strut, the latter junction being located in proximity to the former junction.

9. The stent according to claim 4, wherein
a plurality of the tilting struts are disposed in the first portion at spaced intervals in the circumferential direction, and
the first additional strut and the second additional strut constitute the second portions.

10. The stent according to claim 9, wherein
the first portion includes a plurality of first inclined struts and a plurality of second inclined struts,
the first inclined struts and the second inclined struts are arranged alternately in the circumferential direction, except the tilting strut connected between the first inclined struts or between the second inclined struts,
the first inclined strut extends from one end to other end in such a manner as to be inclined to one side in the circumferential direction at an inclination angle α with respect to the longitudinal axis of the stent,
the second inclined strut extends from one end, which is connected to the other end of the first inclined strut, in a direction opposite to the first inclined strut in such a manner as to be inclined to one side in the circumferential direction at an inclination angle β with respect to the longitudinal axis of the stent, and other end of the second inclined strut is connected to the one end of the next first inclined strut, and
rigidity of the first inclined strut and the second inclined strut constituting the first portion is greater than rigidity of the tilting strut, the first additional strut, and the second additional strut constituting the S-element.

11. The stent according to claim 2, wherein
the tilting strut has a middle part disposed in the first portion, and opposite end portions connected to opposite ends of the middle part and constituting the second portions, and
when the first portion is expanded in the radial direction, the tilting strut tilts in a direction where an inclination angle of the tilting strut with respect to a longitudinal axis of the stent decreases, whereby the spacing between the adjacent first portions increases.

12. The stent according to claim 11, wherein
rigidity of at least the opposite end portions of the tilting strut is lower than rigidity of other struts constituting the first portion.
